(19) United States
(12) Patent Application Publication
(10) Pub. No.: US 2018/0163271 A1
(43) Pub. Date: Jun. 14, 2018

(54) Use of PARP Inhibitors to Treat Breast or Ovarian Cancer Patients Showing a Loss of Heterozygosity

(71) Applicant: Clovis Oncology, Inc., Boulder, CO (US)

(72) Inventor: Kevin Lin, San Mateo, CA (US)

(21) Appl. No.: 15/104,684

(22) PCT Filed: Jan. 14, 2015
(86) PCT No.: PCT/US15/11413

§ 371 Date: Jun. 15, 2016

Related U.S. Application Data
(60) Provisional application No. 61/928,326, filed on Jan. 16, 2014.
(60) Provisional application No. 62/004,424, filed on May 29, 2014.
(60) Provisional application No. 62/039,516, filed on Aug. 20, 2014.
(60) Provisional application No. 62/076,165, filed on Nov. 6, 2014.

(51) Int. Cl. C12Q 1/6886; A61P 35/00; A61K 31/55

(52) U.S. Cl. CPC C12Q 1/6886; A61P 35/00; A61K 31/55; G01N 2800/60; C12Q 2600/156; C12Q 2600/112; C12Q 2600/106

(57) ABSTRACT

In one embodiment, the subject invention relates to a method for treatment of a breast or ovarian cancer patient that includes receiving assay results stating that the patient's tumor exhibits LOH, and administering a PARP inhibitor. In one embodiment, the subject invention comprising: classifying said cancer patient, with the computer system, as being likely to respond to a PARP inhibitor if the data comprises i) one or more deleterious mutations in BRCA1 or BRCA2, or ii) a percentage of the genome having greater than 10 percent LOH as determined by the sum of the lengths of each individual LOH region divided by the total genome length, wherein an LOH region is defined as the presence of homozygosity at multiple contiguous single nucleotides, but excludes whole chromosome or chromosome arm LOH.

### BACKGROUND OF THE INVENTION

In recent years, attention has turned to identifying biomarkers or other measurements in tumor tissue or blood that may predict outcome to various therapeutic interventions. Given the importance of homologous recombination repair genes such as BRCA1 and BRCA2 in maintaining genomic stability, characterizing the extent of genomic instability can lead to identification of homologous recombination defective tumors.

Analyzing a cancer patient's genome for loss of heterozygosity (LOH) has been purported to be a potential marker for genomic instability generally (Walsh S, et al. Genome-wide loss of heterozygosity and uniparental disomy in BRCA1/2-associated ovarian carcinomas. Clin Cancer Res 2008; 14:7645-51). It has been suggested that DNA damaging agents are possible agents for treating patients whose tumors exhibit LOH.

Loss of heterozygosity (LOH) refers to a change from a state of heterozygosity in a normal genome to a homozygous state in a tumor genome (Beroukhim R, et al. Inferring loss-of-heterozygosity from unpaired tumors using high-density oligonucleotide SNP arrays. PLoS Comput Biol 2006; 2:e41). LOH can result from copy loss events such as hemizygous deletions, or from copy neutral events such as uniparental disomy in which the deletion of one allele is accompanied by the gain of the other allele (Walsh S, et al. supra).

However, some types of cancer, including certain lung cancers, are likely to exhibit LOH that is primarily related to environmental factors and may be unrelated to genetic causes of DNA damage. LOH in these cancers are often caused by external causes, not mutations associated with DNA repair mechanisms. These types of cancers are unlikely to benefit from treatment with certain DNA damaging agents, particularly those that rely on synthetic lethality associated with DNA repair as the mechanism of action, such as PARP inhibitors.

Breast cancer, particularly triple negative breast (or basal-like subtype), and ovarian cancer share common features of widespread genomic instability and similar therapeutic approaches such as platinum-based therapies have been suggested (The Cancer Genome Atlas Network. Nature 2012; 490:61-70). In addition, both triple negative and BRCA1/2-associated ovarian cancers have higher frequencies of genome-wide LOH and uniparental disomy (Tuna M, et al. Association between acquired uniparental disomy and homozygous mutations and HER2/ER/PR status in breast cancer. PLoS One 2010; 5:e15094. Walsh S, et al. supra). Therefore, breast and ovarian cancer are the diseases most likely to benefit from identification of LOH and administration of agents that result in synthetic lethality, such as PARP inhibitors.

The subject invention shows for the first time that breast and ovarian cancer cells that exhibit loss of heterozygosity are sensitive to PARP inhibitors, particularly rucaparib.

### SUMMARY OF THE INVENTION

In one embodiment, the subject invention relates to a method for treatment of a breast or ovarian cancer patient that includes receiving assay results stating that the patient's tumor exhibits LOH, and administering a PARP inhibitor. In certain embodiments, the PARP inhibitor is rucaparib.

In one embodiment, the subject invention relates to a method for treatment of a breast or ovarian cancer patient with a PARP inhibitor comprising: a) receiving data from a computer system regarding the tumor of said cancer patient comprising, i) the BRCA1 and BRCA2 mutation status, and ii) the homozygous or heterozygous nature of a plurality of single nucleotides along each chromosome of the genome; b) classifying said cancer patient, with the computer system, as being likely to respond to a PARP inhibitor if the data comprises i) one or more deleterious mutations in BRCA1 or BRCA2, or ii) a percentage of the genome having greater than 10 percent LOH as determined by the sum of the lengths of each individual LOH region divided by the total genome length, wherein an LOH region is defined as the presence of homozygosity at multiple contiguous single nucleotides, but excludes whole chromosome or chromosome arm LOH; and c) administering a therapeutically effective amount of a PARP inhibitor to said cancer patient whose classification meets the criterion of step b).

In one embodiment, LOH is determined by using a hidden Markov model-based method to identify LOH in the tumor samples.

In one embodiment, LOH is determined by using the Allele-Specific Copy number Analysis of Tumor (ASCAT) method to identify LOH in the tumor samples.

### BRIEF DESCRIPTION OF THE FIGURES

FIG. 1 is an overview of the bioinformatics analysis workflow to determine the percentage of genome with LOH in breast cancer cell lines.

FIG. 2 plots the correlation between the percentage of genome with LOH and rucaparib sensitivity in breast cancer cell lines. Triple-negative breast cancer (TNBC) and non-TNBC cell lines are indicated with filled and unfilled markers, respectively.

FIG. 3 is a receiver operating characteristic (ROC) curve for the percentage of genome with LOH in predicting rucaparib sensitivity in TNBC cell lines. Fitted ROC area under the curve=0.853.

FIG. 4 defines the cut-off for the percentage of genome with LOH to predict rucaparib sensitivity in TNBC cell lines. Vertical dashed line: percentage of genome with LOH set at the cut-off of 20%. Horizontal dashed line: rucaparib sensitive cell lines defined as 2.05 μM or less.

FIG. 5 is an overview of the bioinformatics analysis workflow to determine the percentage of genome with LOH in high-grade serous ovarian tumors.

FIG. 6 is a histogram showing the wide range of percentage of genome with LOH in high-grade serous ovarian tumors. The vertical dashed line indicates the median percentage of genome with LOH.

FIG. 7 is a Kaplan-Meier plot of overall survival following platinum-based chemotherapy in patients with high (solid line) vs low (dashed line) genomic LOH tumors. Markers indicate censored data points.

FIG. 8 is a Kaplan-Meier plot of overall survival following platinum-based chemotherapy in HRD-positive (solid line) vs HRD-negative patients (dashed line). Markers indicate censored data points.

FIG. 9 is a Kaplan-Meier plot of overall survival following platinum-based chemotherapy in HRD-positive (solid line) vs HRD-negative patients (dashed line). Markers indicate censored data points.

FIG. 10 is an overview of the bioinformatics analysis workflow to determine the percentage of genome with LOH in FFPE ovarian tumors in a phase I clinical trial.

FIG. 11 is an overview of the bioinformatics analysis workflow to determine the percentage of genome with LOH in FFPE high-grade ovarian tumors from a phase II clinical trial.

FIG. 12 is a waterfall plot of the best target lesion response to rucaparib using the RECIST 1.1 criteria at time point A. The y-axis is the percentage change of the target tumor lesion from baseline to post-rucaparib treatment. The upper and lower dash lines indicate the thresholds of 20% increase (progressive disease) and 30% decrease (partial response) from baseline, respectively. The HRD status is determined for all patients except one case (labeled as “Unknown”) that failed genomic LOH analysis due to low tumor content.

FIG. 13 is a waterfall plot of the best target lesion response to rucaparib using the RECIST 1.1 criteria at time point B. The y-axis is the percentage change of the target tumor lesion from baseline to post-rucaparib treatment. The upper and lower dash lines indicate the thresholds of 20% increase (progressive disease) and 30% decrease (partial response) from baseline, respectively. The HRD status is determined for all patients except one case (labeled as “Unknown”) that failed genomic LOH analysis due to low tumor content.

FIG. 14 is a waterfall plot of the best target lesion response to rucaparib using the RECIST 1.1 criteria for patients in the BRCA subgroup at time point C. The y-axis is the percentage change of the target tumor lesion from baseline to post-rucaparib treatment. The upper and lower dash lines indicate the thresholds of 20% increase (progressive disease) and 30% decrease (partial response) from baseline, respectively. Patients with CA-125 response have patterned bars. Patients still on rucaparib treatment are marked with “+”.

FIG. 15 is a waterfall plot of the best target lesion response to rucaparib using the RECIST 1.1 criteria for patients in the Non-BRCA/LOH+ subgroup at time point C. The y-axis is the percentage change of the target tumor lesion from baseline to post-rucaparib treatment. The upper and lower dash lines indicate the thresholds of 20% increase (progressive disease) and 30% decrease (partial response) from baseline, respectively. Patients with CA-125 response have patterned bars. Patients still on rucaparib treatment are marked with “+”.

FIG. 16 is a waterfall plot of the best target lesion response to rucaparib using the RECIST 1.1 criteria for patients in the Non-BRCA/LOH− subgroup at time point C. The y-axis is the percentage change of the target tumor lesion from baseline to post-rucaparib treatment. The upper and lower dash lines indicate the thresholds of 20% increase (progressive disease) and 30% decrease (partial response) from baseline, respectively. Patients with CA-125 response have patterned bars. Patients still on rucaparib treatment are marked with “+”.

### DETAILED DESCRIPTION OF THE INVENTION

It is a main objective of the present invention to treat breast and ovarian cancer patients that have demonstrated DNA damage, based on the presence of LOH, with a PARP inhibitor, particularly rucaparib. The presence of LOH in a breast or ovarian tumor helps guide a health practitioner's treatment choice.

The subject invention relates to a method for treatment of a breast or ovarian cancer patient with a PARP inhibitor comprising: a) receiving data from a computer system regarding the tumor of said cancer patient comprising, i) the BRCA1 and BRCA2 mutation status, and ii) the homozygous or heterozygous nature of a plurality of single nucleotides along each chromosome of the genome; b) classifying said cancer patient, with the computer system, as being likely to respond to a PARP inhibitor if the data comprises i) one or more deleterious mutations in BRCA1 or BRCA2, or ii) a percentage of the genome having greater than 10 percent LOH as determined by the sum of the lengths of each individual LOH region divided by the total genome length, wherein an LOH region is defined as the presence of homozygosity at multiple contiguous single nucleotides, but excludes whole chromosome or chromosome arm LOH; and c) administering a therapeutically effective amount of a PARP inhibitor to said cancer patient whose classification meets the criterion of step b).

To assist in the understanding, explanation and practice of the subject invention, the definitions of terms are provided throughout the Detailed Description.

As used herein, “loss of heterozygosity” or “LOH” refers to a change from a state of heterozygosity in a normal genome to a homozygous state in a tumor genome (Beroukhim R, et al. Inferring loss-of-heterozygosity from unpaired tumors using high-density oligonucleotide SNP arrays. PLoS Comput Biol 2006; 2:e41, which is incorporated by reference herein in its entirety). Determination of LOH can be accomplished using methods known in the art. LOH can be determined using data generated by array comparative genomic hybridization (aCGH), SNP array, next generation sequencing, or other methods. Determination of LOH can be performed by any method known in the art and includes, but is not limited to, subjective analysis by visual inspection, and automated systems coupled with algorithms. One embodiment for determining LOH is the Hidden Markov Model-based method described in Beroukhim, supra. Another embodiment for determining LOH is the Allele-Specific Copy number Analysis of Tumor (ASCAT) method (Van Loo, et al. Allelic-specific copy number analysis of tumors. Proc Natl Acad Sci U.S.A. 2010; 107:16910-5).

LOH is also sometimes referred to as genomic scarring or uniparental disomy (UDP).

“LOH region” refers to a region of a chromosome that contains at least one region of loss of heterozygosity. An LOH region is defined as the presence of homozygosity at multiple contiguous single nucleotides, but excludes whole chromosome, chromosome arm LOH, and X and Y chromosomes.

“Presence of homozygosity at multiple contiguous single nucleotides” refers to the essentially homozygous nature of an LOH region.

“High percentage of genome with LOH” refers to a percentage of the tumor genome having greater than about 10 percent LOH as determined by the sum of the lengths of each individual LOH region divided by the total genome length. In some embodiments, the percentage of the genome having LOH as determined by the sum of the lengths of each individual LOH region divided by the total genome length is greater than about 11 percent, greater than about 12 percent, greater than about 13 percent, greater than about 14 percent, greater than about 15 percent, greater than about 16 percent, greater than about 17 percent, greater than about 18 percent, greater than about 19 percent, or greater than about 20 percent.

“PARP Inhibitor” refers to any compound whose primary activity is the inhibition of PARP activity, including PARP1 and PARP2. PARP inhibitors include rucaparib, olaparib, veliparib, iniparib, BMN-673, niraparib. Rucaparib is the preferred PARP inhibitor.

“Breast cancer” refers to cancer originating from the breast tissue, such as the ducts (ductal carcinomas) or lobules (lobular carcinomas).

“Triple negative breast cancer” refers to the lack of expression of three types of receptors on the surface of tumor cells: estrogen receptor (ER), progesterone receptor (PR), and HER2. Triple negative breast cancer is highly overlapped with the molecular subtype of breast cancer termed basal-like, defined by gene expression profiles.

“Ovarian cancer” refers to cancer originating from the ovary, such as the epithelial tissue (epithelial ovarian cancer). High-grade serous ovarian cancer is the most common subtype and displays widespread genomic instability, indicating likely a defect in homologous recombination (Bowtell D D, Nat Rev Cancer 2010; 10: 803-8).

“Homologous recombination defect” refers to the inability of cells to undergo repair of the DNA with double-strand breaks due to aberrations in DNA repair genes.

“Deleterious BRCA1/2 mutations” are well-known to one of ordinary skill in the art and refer to all protein-truncating mutations (frameshift insertion/deletion or nonsense), functional missense mutations (e.g. BRCA1 C61G mutation), and homozygous deletions of BRCA1/2 genes (Cancer Genome Atlas Research Network. Integrated genomic analyses of ovarian carcinomas. Nature 2011; 474:609-15).

“HRD-positive tumors” refers to tumors containing either deleterious BRCA1/2 mutations or tumors with high percentage of genome with LOH. HRD-positive tumors are most likely to be sensitive to agents to such as PARP inhibitors and platinum. Patients having HRD-positive tumors treated with a PARP inhibitor, such as rucaparib, are most likely to have a significantly longer overall survival than patients having HRD-negative tumors.

“HRD-negative tumors” refers to tumors containing no deleterious BRCA1/2 mutation and without a high percentage of genome with LOH.

“Patient” includes mammals, for example, humans. Patients include those having a disease, those suspected of having a disease, and those in which the presence of a disease is being assessed.

“Treating” or “treatment” of a disease refers to arresting or substantially slowing the growth of breast or ovarian cancer cells, or at least one of the clinical symptoms of these cells. In certain embodiments, “treating” or “treatment” refers to arresting or reducing at least one physical parameter of the cancer, which may or may not be discernible by the patient. In certain embodiments, “treating” or “treatment” refers to inhibiting or controlling the cancer, either physically (e.g., stabilization of a discernible symptom), physiologically (e.g., stabilization of a physical parameter), or both.

“Therapeutically effective amount” refers to the amount of a compound that, when administered to a subject for treating breast or ovarian cancer, is sufficient to affect such treatment of the cancer. The “therapeutically effective amount” may vary depending, for example, on the PARP inhibitor selected, the stage of the cancer, the age, weight and/or health of the patient and the judgment of the prescribing physician. An appropriate amount in any given instance may be readily ascertained by those skilled in the art or capable of determination by routine experimentation.

A “sample” or “biological sample” is a biological specimen containing genomic DNA, RNA (including mRNA), protein, or combinations thereof, obtained from a subject. Examples include, but are not limited to, chromosomal preparations, peripheral blood, urine, saliva, tissue biopsy, surgical specimen, bone marrow, amniocentesis samples and autopsy material. In one example, a sample includes genomic DNA or RNA. In some examples, the sample is a cytogenetic preparation, for example which can be placed on microscope slides. In particular examples, samples are used directly, or can be manipulated prior to use, for example, by fixing (e.g., using formalin).

Methods described herein can be extended to a variety of cancers. Preferred cancers are breast, ovarian, and pancreatic cancer. In some instances, cancer can be a metastatic cancer. Examples of additional cancers related to the methods described herein include, but are not limited to, sarcoma, prostate cancer, colon cancer (such as a colon carcinoma, including small intestine cancer), glioma, leukemia, liver cancer, melanoma (e.g., metastatic malignant melanoma), acute myeloid leukemia, kidney cancer, bladder cancer, renal cancer (e.g., renal cell carcinoma), glioblastoma, brain tumors, chronic or acute leukemias including acute lymphocytic leukemia (ALL), adult T-cell leukemia (T-ALL), chronic myeloid leukemia, acute lymphoblastic leukemia, chronic lymphocytic leukemia, lymphomas (e.g., Hodgkin's and non-Hodgkin's lymphoma, lymphocytic lymphoma, primary CNS lymphoma, T-cell lymphoma, Burkitt's lymphoma, anaplastic large-cell lymphomas (ALCL), cutaneous T-cell lymphomas, nodular small cleaved-cell lymphomas, peripheral T-cell lymphomas, Lennert's lymphomas, immunoblastic lymphomas, T-cell leukemia/lymphomas (ATLL), entroblastic/centrocytic (cb/cc) follicular lymphomas cancers, diffuse large cell lymphomas of B lineage, angioimmunoblastic lymphadenopathy (AILD)-like T cell lymphoma and HIV associated body cavity based lymphomas), embryonal carcinomas, undifferentiated carcinomas of the rhino-pharynx (e.g., Schmincke's tumor), Castleman's disease, Kaposi's Sarcoma, multiple myeloma, Waldenstrom's macroglobulinemia and other B-cell lymphomas, nasopharangeal carcinomas, bone cancer, skin cancer, cancer of the head or neck, cutaneous or intraocular malignant melanoma, uterine cancer, rectal cancer, cancer of the anal region, stomach cancer, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), tumor angiogenesis, spinal axis tumor, brain stem glioma, pituitary adenoma, epidermoid cancer, squamous cell cancer, or environmentally induced cancers including those induced by asbestos, e.g., mesothelioma. In another embodiment, methods described herein can be useful for treating a combination of two or more types of cancer. In some aspects the methods are useful to treat individual patients diagnosed with cancer.

The invention having now been described by way of written description, those of skill in the art will recognize that the invention can be practiced in a variety of aspects and that the foregoing description and examples below are for purposes of illustration and not limitation of the claims that follow.

### EXAMPLES

While alternative aspects have been shown and described herein, it will be obvious to those skilled in the art that such aspects are provided by way of example only. Numerous variations, changes, and substitutions will occur to those skilled in the art without departing from the invention. It should be understood that various alternatives to the aspects of the invention described herein can be employed in practicing the invention. It is intended that the following claims define the scope of the invention and that methods and structures within the scope of these claims and their equivalents be covered thereby.

### Example 1

### Rucaparib Sensitive Breast Cancer Cells Exhibit Genomic LOH

### Rucaparib Sensitive Cells

Rucaparib sensitivity data in a large panel of human cancer cells lines were generated using a high throughput growth inhibition assay. Briefly, cells were plated into 24-well tissue culture plates at a cell density of 5 to 20×10³ cells. Rucaparib was treated in concentrations ranging from 0.005 to 10 μM. Viable cells were counted on day 1 and day 6 of rucaparib treatment using a Beckman Coulter Z2 particle counter. Growth inhibition was calculated as a function of the number of generations inhibited in the presence of rucaparib versus the number of generations over the same time course in the absence of rucaparib. Dose response curves were generated and the half maximal effective concentration (EC50) values for growth inhibition were calculated for each cell line. Some of the most sensitive cell lines found in the high throughput screen were breast cancer cell lines (Table 1).

TABLE 1
Rucaparib sensitivity in 36 breast cancer cell lines used in LOH
analyses. Cell lines are sorted from the most to least sensitive
to rucaparib (EC50 values). Triple negative status is based on
previously published report of TNBC cell lines (Lehmann B D, Bauer
J A, Chen X, et al. Identification of human triple-negative breast
cancer subtypes and preclinical models for selection of targeted
therapies. J Clin Invest 2011; 121: 2750-67).

| | Triple-negative | EC50 |
|---|---|---|
| Cell line | status | (μM) |
| HCC1395 | TNBC | 0.0545 |
| HCC202 | Non-TNBC | 0.201 |
| HCC1187 | TNBC | 0.37 |
| MDAMB436 | TNBC | 0.386 |
| ZR7530 | Non-TNBC | 0.65 |
| HCC38 | TNBC | 0.689 |
| HCC1500 | Non-TNBC | 0.757 |
| HCC1569 | Non-TNBC | 0.945 |
| MDAMB361 | Non-TNBC | 1.36 |
| UACC893 | Non-TNBC | 1.55 |
| MDAMB157 | TNBC | 1.72 |
| HCC2218 | Non-TNBC | 1.86 |
| MDAMB453 | TNBC | 1.91 |
| KPL1 | Non-TNBC | 1.94 |
| MDAMB468 | TNBC | 2.05 |
| T47D | Non-TNBC | 2.32 |
| EFM19 | Non-TNBC | 2.6 |
| HCC1806 | TNBC | 3.2 |
| HCC70 | TNBC | 3.72 |
| HCC1419 | Non-TNBC | 4.14 |
| MDAMB415 | Non-TNBC | 4.19 |
| DU4475 | TNBC | 4.57 |
| ZR751 | Non-TNBC | 4.63 |
| EFM192A | Non-TNBC | 5.68 |
| MCF7 | Non-TNBC | 7.01 |
| UACC812 | Non-TNBC | 7.01 |
| HCC1143 | TNBC | 7.17 |
| SKBR3 | Non-TNBC | 7.97 |
| BT474 | Non-TNBC | 8.75 |
| CAMA1 | Non-TNBC | 9.13 |
| BT549 | TNBC | 9.75 |
| MDAMB231 | TNBC | 9.79 |
| BT20 | TNBC | 10 |
| CAL51 | TNBC | 10 |
| HCC1937 | TNBC | 10 |
| HCC1954 | Non-TNBC | 10 |

### LOH Analysis

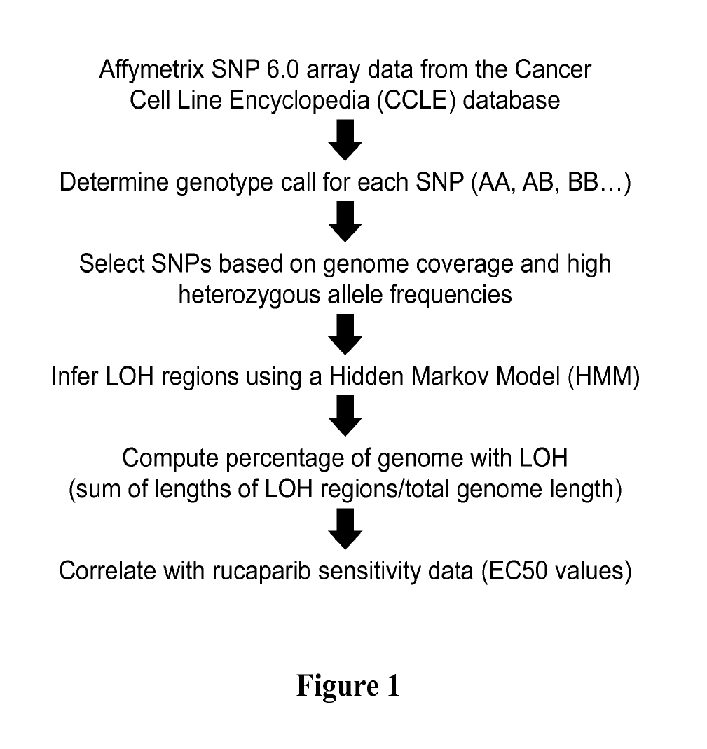

The rucaparib sensitive breast cancer cell lines found in the high throughput screen were used to demonstrate the utility of the percentage genome with LOH in predicting rucaparib sensitivity. For each cell line, LOH analysis of Affymetrix SNP 6.0 array was performed to determine the percentage of genome with LOH. An overview of the bioinformatic analysis workflow is outlined in FIG. 1.

Briefly, Affymetrix SNP 6.0 array intensity data (.CEL files) were downloaded from the publicly available Cancer Cell Line Encyclopedia database (CCLE; http://www.broadinstitute.org/ccle/home, 2012-04-05 version). SNP genotype calls (.CHP files) were generated from the array intensity data using the Birdseed v2 algorithm with the default confidence threshold of 0.1 in Affymetrix Genotyping Console. For LOH inference, 2998 SNPs on the Affymetrix SNP 6.0 array were selected based on genome coverage and high heterozygous allele frequencies in the HapMap western European population. Since there is no reference normal sample for the panel of cancer cell lines, LOH regions were inferred using unpaired analysis with Hidden Markov Model (HMM) as previously described (Beroukhim R, Lin M, Park Y, et al. Inferring loss-of-heterozygosity from unpaired tumors using high-density oligonucleotide SNP arrays. PLoS Comput Biol 2006; 2:e41). Default parameters were used for the unpaired analysis: expected genotype error rate of 0.01 and heterozygous frequency of 0.5. LOH regions spanning across the whole chromosome were excluded from the analysis as well as exclusion of X and Y chromosomes. Chromosomes 13, 14, 15, 21, and 22 have short heterochromatic p chromosome arms that lack SNP representation, so LOH regions spanning the q chromosome arms were excluded as well. The percentage of the genome with LOH was determined by the sum of the lengths of each individual LOH region divided by the total genome length with SNP coverage (2.77E+09 base pairs). For example, for cell line HCC1395, after excluding whole chromosome LOH regions, the sum of all remaining LOH regions is 1.122E+09 base pairs, and when divided by 2.77E+09 base pairs results in 40.5% of genome with LOH.

### Statistics

Spearman's rank test was performed to determine the significance of correlation between percentage of genome with LOH and rucaparib sensitivity (EC50). Fitted receiver operating characteristic (ROC) curve was generated using the percentage of genome with LOH as a continuous rating scale as previously described (Eng J. Receiver operating characteristic analysis: a primer. Acad Radiol 2005; 12:909-16). Fisher's exact test was performed to determine the significance of the 2×2 contingency table for predicting rucaparib sensitivity.

### Results

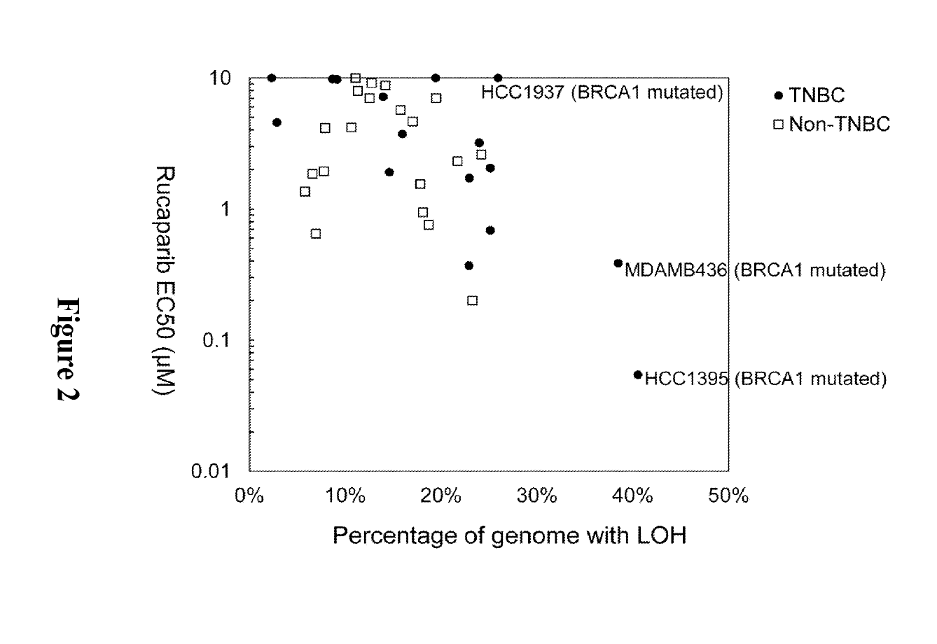

An association between higher percentage of genome with LOH and increased rucaparib sensitivity (i.e. lower EC50 values) was found in the panel of 36 breast cancer cell lines (p=0.03) (FIG. 2). Furthermore, 16 of the 36 breast cancer cell lines in the screen are triple negative breast cancer (TNBC), and the association is significantly correlated in TNBC cell lines (p=0.02). Three of the TNBC cell lines contain deleterious BRCA1 mutations, and these cell lines have the highest percentages of genome with LOH in the breast cancer cell line panel (HCC1395—40.5%, MDAMB436—38.5%, HCC1937—25.9%). As expected for cells with deleterious BRCA mutations being homologous recombination defective (HRD), both HCC1395 and MDAMB436 are highly sensitive to rucaparib (<0.5 μM). Despite having a deleterious BRCA1 mutation, HCC1937 is not sensitive to rucaparib, which is likely due to resistance mechanisms to DNA damaging agents.

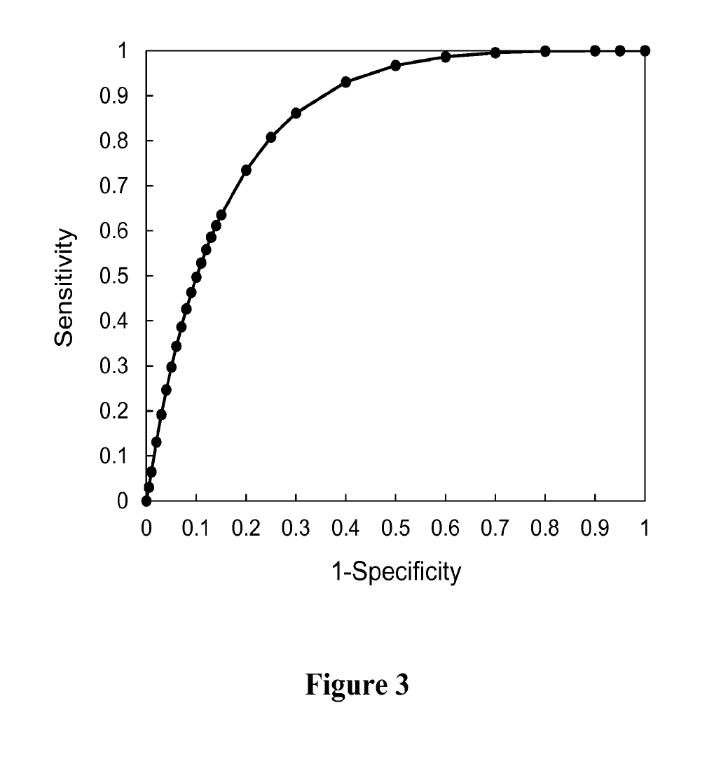

To test the potential diagnostic utility of the percentage of genome with LOH in predicting rucaparib sensitivity in TNBC, a receiver operating characteristic (ROC) analysis was performed. Since TNBC cell lines HCC1395, MDA-MB-436, and MDA-MB-468 are known to be sensitive to rucaparib, the threshold for defining a rucaparib sensitive cell line is set at the highest EC50 value of these cell lines; EC50 of MDA-MB-468 is 2.05 μM. Using this criterion, a ROC curve was generated and indicated that the percentage of genome with LOH can be useful in predicting rucaparib sensitivity (FIG. 3, fitted ROC area under the curve=0.853).

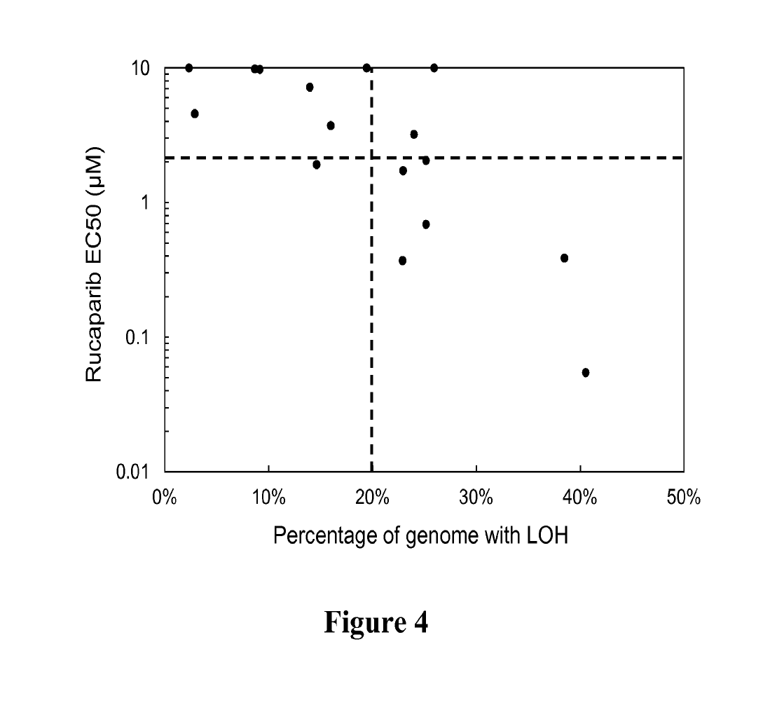

In addition, a cut-off for the percentage of genome with LOH can be set to predict whether a TNBC cell line is likely to respond to rucaparib. For example, if the cut-off is set at 20% of genome with LOH, the sensitivity and specificity for predicting rucaparib response in TNBC cell lines are 86% (6 of 7 rucaparib-sensitive cell lines had >20% of genome with LOH) and 78% (7 of 9 rucaparib-resistant cell lines had <20% of genome with LOH), respectively (FIG. 4, Table 2).

TABLE 2
2x2 contingency table for the use of percentage
of genome with LOH to predict rucaparib sensitivity.
Fisher's exact test: p = 0.04.

| | Rucaparib sensitive | Rucaparib resistant |
|---|---|---|
| | (EC50 < 2.05 μM) | (EC50 ≥ 2.05 μM) |
| Predicted rucaparib sensitive | 6 | 2 |
| (≥20% of genome with LOH) | | |
| Predicted rucaparib resistant | 1 | 7 |
| (<20% of genome with LOH) | | |

The cut-offs for the percentage of genome with LOH described here applies to TNBC cell lines profiled using Affymetrix SNP6.0 arrays. However, the cut-offs can be adjusted based on the sample type studied (e.g. cell line vs tumor) and genomic analysis platform used (e.g. Affymetrix SNP 6.0 arrays vs next generation sequencing of targeted sequencing of SNPs). Furthermore, the cut-offs may be tailored for different cancer indications, such as high-grade serous ovarian cancer which is likely to also display genomic instability and LOH.

### Example 2—Ovarian Cancer Patients with Tumors Exhibiting Genomic LOH Benefit from Platinum-Based Treatment

### High-Grade Serous Ovarian Tumors

The Cancer Genome Atlas (TCGA) project performed genomic analysis of 316 high-grade serous ovarian tumors (Cancer Genome Atlas Research Network. Integrated genomic analyses of ovarian carcinomas. Nature 2011; 474:609-15). Samples were collected from newly diagnosed patients with ovarian serous adenocarcinoma who were undergoing surgical resection and had received no prior treatment. As standard therapy, patients were then treated with platinum-based chemotherapy and overall survival was recorded (the interval from the date of initial surgical resection to the date of last known contact or death). Patients with platinum free interval (interval from the date of last primary platinum treatment to the date of progression) of six months or greater are defined as platinum-sensitive. Next generation sequencing of tumors identified deleterious BRCA1/2 mutations, which include all protein-truncating mutations (frameshift insertion/deletion or nonsense), functional missense mutations (e.g. BRCA1 C61G mutation), and homozygous deletions of BRCA1/2 genes (Cancer Genome Atlas Research Network. Integrated genomic analyses of ovarian carcinomas. Nature 2011; 474:609-15).

### LOH Analysis

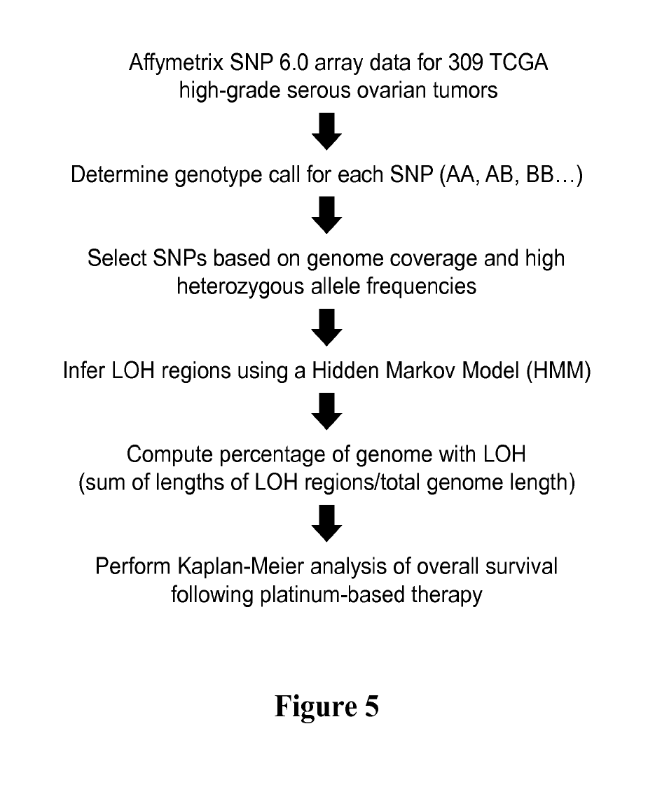

The high-grade serous ovarian tumors in TCGA study were used to demonstrate the utility of the percentage genome with LOH in predicting overall survival following platinum-based chemotherapy. For each tumor, LOH analysis of Affymetrix SNP 6.0 array was performed to determine the percentage of genome with LOH. An overview of the bioinformatic analysis workflow is outlined in FIG. 5.

Briefly, Affymetrix SNP 6.0 array intensity data (.CEL files) were downloaded from the publicly available TCGA database (https://tcga-data.nci.nih.gov/tcga/tcgaDownload.jsp, 2010-06-05 version). SNP genotype calls (.CHP files) were generated from the array intensity data using the Birdseed v2 algorithm with the default confidence threshold of 0.1 in Affymetrix Genotyping Console. For LOH inference, 2998 SNPs on the Affymetrix SNP 6.0 array were selected based on genome coverage and high heterozygous allele frequencies in the HapMap western European population. Since there is no reference normal sample for the panel of cancer cell lines, LOH regions were inferred using unpaired analysis with Hidden Markov Model (HMM) as previously described (Beroukhim R, Lin M, Park Y, et al. Inferring loss-of-heterozygosity from unpaired tumors using high-density oligonucleotide SNP arrays. PLoS Comput Biol 2006; 2:e41). Default parameters were used for the unpaired analysis: expected genotype error rate of 0.01 and heterozygous frequency of 0.5. LOH regions spanning across the whole chromosome were excluded from the analysis as well as exclusion of X and Y chromosomes. Chromosomes 13, 14, 15, 21, and 22 have short heterochromatic p chromosome arms that lack SNP representation, so LOH regions spanning the q chromosome arms were excluded as well. The percentage of the genome with LOH was determined by the sum of the lengths of each individual LOH region divided by the total genome length with SNP coverage.

### Statistics

Kaplan-Meier survival analysis was performed to determine the median and log-rank p-value of the difference in overall survival of patients with high versus low percentage of genome with LOH. Cox proportional hazards models was used to calculate the hazard ratios and multivariate analysis.

### Results

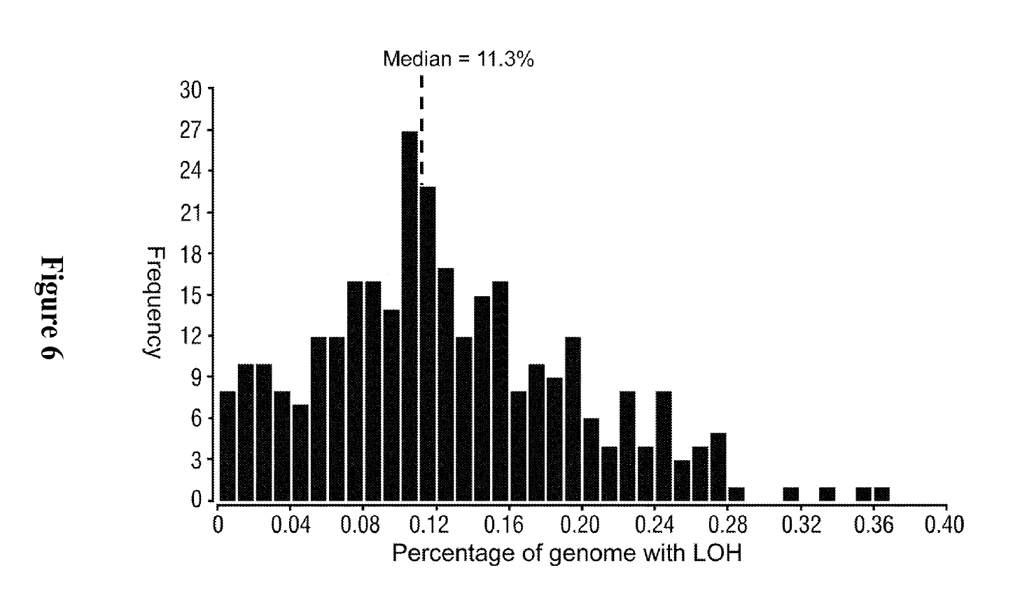

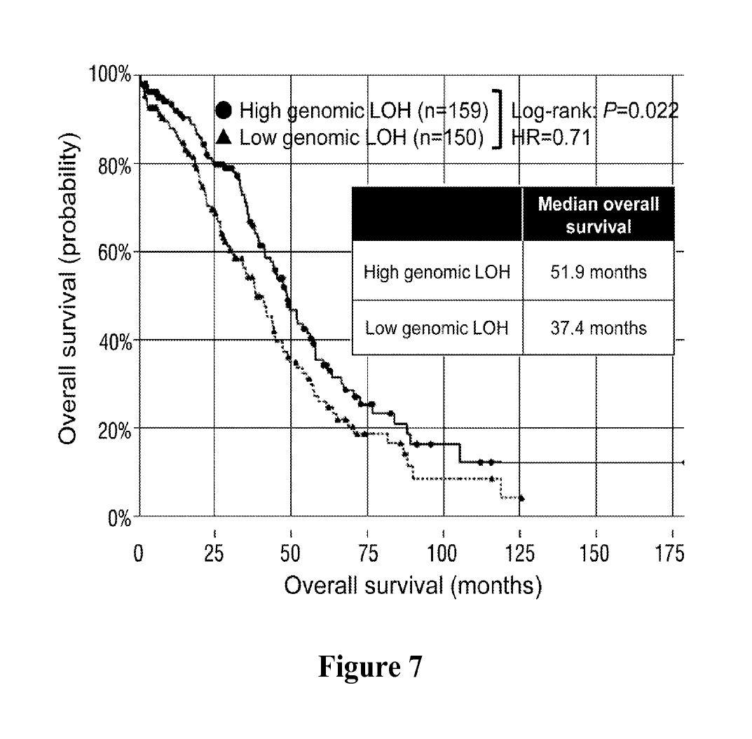

High-grade serous ovarian tumors from the TCGA study displayed a wide range of percentage of genome with LOH, with the median at 11.3% (FIG. 6). Patients can be classified into the high genomic LOH group if the percentage of genome with LOH is greater than the median and into the low genomic LOH group if lower than the median. Significant separation of Kaplan-Meier overall survival curves was found between high vs low genomic LOH (p=0.022, hazard ratio=0.71, FIG. 7), indicating the potential utility of the percentage genome with LOH in predicting overall survival following platinum-based chemotherapy. Multivariate analysis of factors impacting overall survival found that the percentage of genome with LOH is an independent predictor of overall survival following platinum-based chemotherapy (p=0.035, hazard ratio=0.72, Table 3).

TABLE 3
Cox multivariate analysis of factors impacting overall survival
following platinum-based chemotherapy in ovarian tumors

| | | | Hazard | |
|---|---|---|---|---|
| | | | ratio | p-value |
| Percentage of genome | High genomic LOH | | 0.72 | 0.035 |
| with LOH | Low genomic LOH | | 1 | |
| BRCA status | BRCA | | 0.49 | 0.00033 |
| | Non-BRCA | | 1 | |
| Tumor stage | II | | 0.58 | 0.27 |
| | III | | 0.95 | 0.80 |
| | IV | | 1 | |
| Tumor grade | G2 | | 0.72 | 0.22 |
| | G3 | | 1 | |
| Tumor residual disease | No macroscopic disease | | 0.47 | 0.010 |
| | 1-10 | mm | 0.87 | 0.48 |
| | 11-20 | mm | 0.75 | 0.44 |
| | >20 | mm | 1 | |

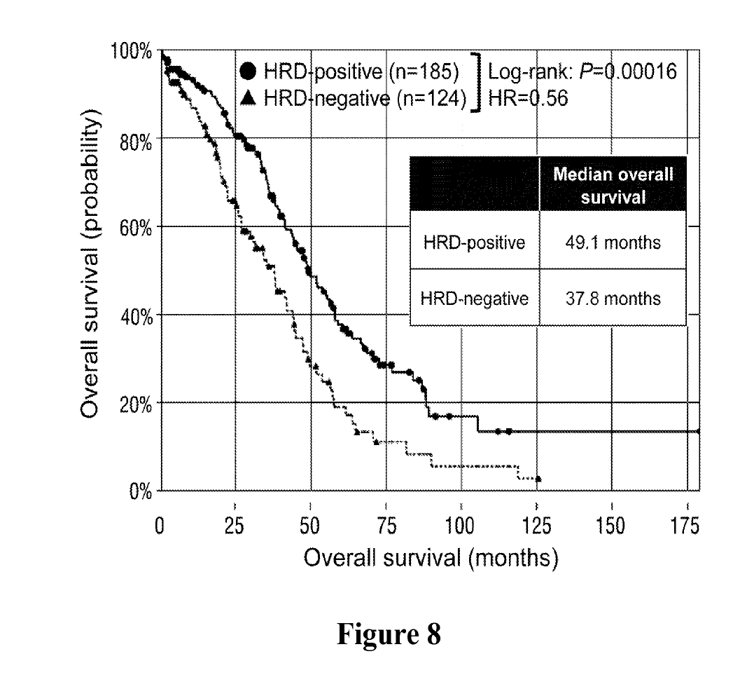

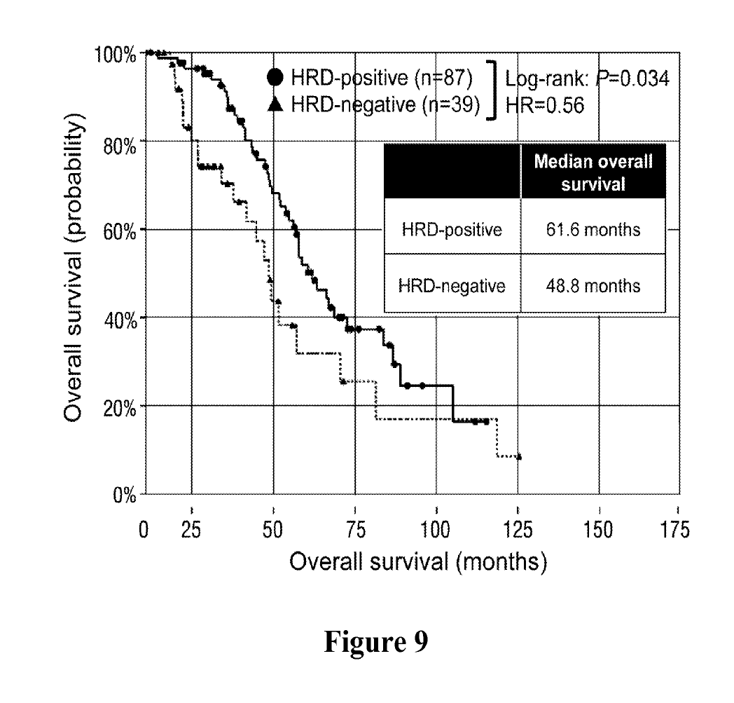

Since patients with tumors containing BRCA1/2 mutations are known to be sensitive to DNA damaging agents and BRCA mutations are drivers of HRD, these patients can be grouped together with patients who have tumors with high percentage of genome with LOH to form a group called HRD-positive patients who are most likely to be sensitive to platinum and rucaparib. Consistent with this hypothesis, HRD-positive patients were found to have significantly longer overall survival than HRD-negative (p=0.00016, hazard ratio=0.56, FIG. 8). In addition, the difference in overall survival between HRD-positive and HRD-negative was also found in platinum-sensitive patients (p=0.034, hazard ratio=0.56, FIG. 9).

### Example 3—Ovarian Cancer Patients with Tumors Exhibiting Genomic LOH Benefit from Rucaparib Treatment

Next-Generation Sequencing of Ovarian Tumors from Patients

Archival tumor tissue samples were optionally collected from patients for genomic analysis. To determine the maximum tolerated dose and recommended Phase II dose, patients were placed in dose-escalation cohorts for oral administration of rucaparib on a continuous daily basis. Antitumor activity of rucaparib was evaluated based on Response Evaluation Criteria in Solid Tumors (RECIST) Version 1.1. In addition, the concentration of cancer antigen-125 (CA-125) in blood was measured as a biomarker for ovarian cancer. Local BRCA1/2 testing results were based on sequencing of BRCA1/2 genes from blood samples (peripheral blood mononuclear cells).

### LOH Analysis

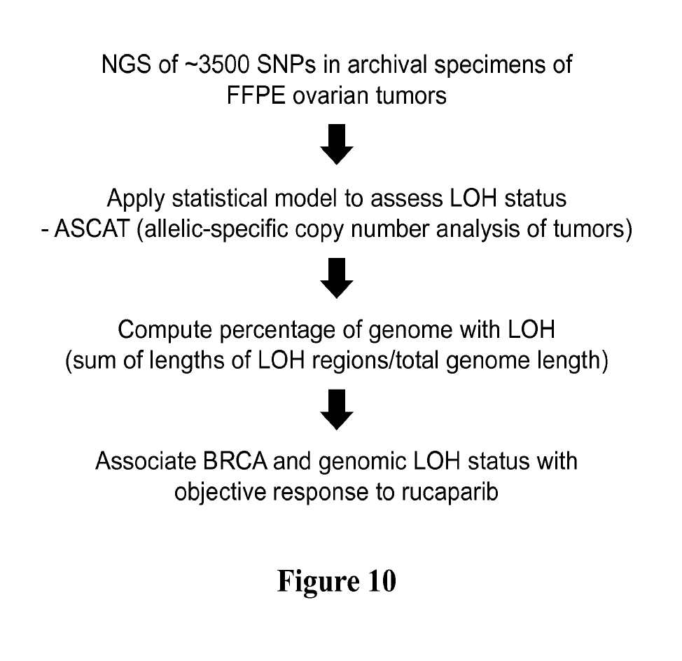

An overview of the bioinformatic analysis workflow is outlined in FIG. 10. Briefly, formalin-fixed paraffin-embedded (FFPE) tumor tissue samples were sequenced using Foundation Medicine's T5 next-generation sequencing (NGS) assay, which includes sequencing of ˜3500 SNPs with good genome coverage and high heterozygous allele frequencies. A statistical model, Allelic-Specific Copy Number Analysis of Tumors (ASCAT), was used to assess LOH status of the sequenced SNPs. LOH regions spanning across the whole chromosome were excluded from the analysis as well as exclusion of X and Y chromosomes. Chromosomes 13, 14, 15, 21, and 22 have short heterochromatic p chromosome arms that lack SNP representation, so LOH regions spanning the q chromosome arms were excluded as well. The percentage of the genome with LOH was determined by the sum of the lengths of each individual LOH region divided by the total genome length with SNP coverage.

### Results

Genomic LOH analysis of five FFPE ovarian tumors found that all tumors had a high percentage of genome with LOH, greater than the median of 11.3% identified in TCGA high-grade serous tumors as shown in Example 2. Furthermore, since these tumors were from patients who all derived clinical benefit from rucaparib treatment (stable or no measurable disease), suggesting that patients with a high percentage of genome with LOH may benefit from rucaparib treatment (Table 4). The patient with the highest percentage of genome with LOH (39.3%) responded to rucaparib treatment based on the concentration of CA-125 cancer antigen.

TABLE 4
Clinical benefit of rucaparib for ovarian cancer
patients with high percentage of genome with LOH

| | | | | Percentage |
|---|---|---|---|---|
| Subject | | Best response | Local BRCA | of genome |
| ID | Cohort | to rucaparib | testing result | with LOH |
| 04-0501 | 300 mg QD | Stable disease | BRCA1 | 25.1% |
| 03-0702 | 300 mg QD | Stable disease | BRCA2 | 20.7% |
| 04-0901 | 360 mg BID | Stable disease, | BRCA1 | 39.3% |
| | | CA-125 response | | |
| 04-0902 | 360 mg BID | Stable disease | BRCA1 | 16.0% |
| 02-1101 | 600 mg BID | No measurable | BRCA2 | 24.4% |
| | | disease | | |

### Example 4—Non-BRCA Patients with Tumors Exhibiting Genomic LOH Also Benefit from Rucaparib Treatment

Next-Generation Sequencing of High-Grade Ovarian Tumors from Patients

Patients with platinum-sensitive, relapsed, high-grade ovarian cancer are treated with oral administration of rucaparib at the recommended Phase 2 dose of 600 mg BID (twice a day). Antitumor activity of rucaparib was evaluated based on Response Evaluation Criteria in Solid Tumors (RECIST) Version 1.1 as well as Gynecologic Cancer Intergroup (GCIG) CA-125 response. Formalin-fixed paraffin-embedded (FFPE) tumor tissue samples were sequenced using Foundation Medicine's T5 next-generation sequencing (NGS) assay, which includes sequencing of 287 cancer-associated genes (including BRCA1/2) as well as ˜3500 SNPs with good genome coverage and high heterozygous allele frequencies. Deleterious BRCA1/2 mutations detected in the tumor tissue (both germline and somatic) include protein-truncating mutations, known missense mutations, and homozygous deletions of the BRCA1/2 genes.

### LOH Analysis

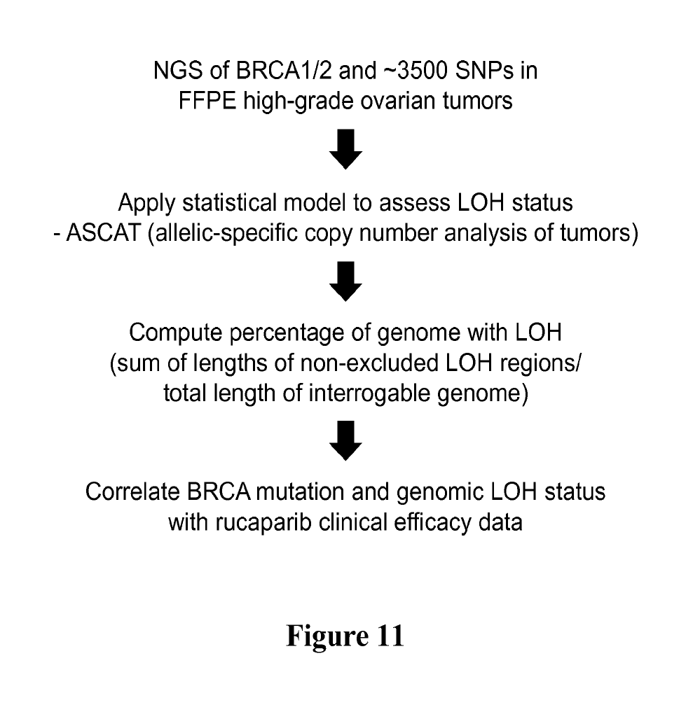

An overview of the bioinformatic analysis workflow is outlined in FIG. 11. Briefly, a statistical model, Allelic-Specific Copy Number Analysis of Tumors (ASCAT), was used to assess LOH status of the sequenced SNPs. LOH regions spanning across the whole chromosome or chromosome arm as well as LOH regions on the X and Y chromosomes were excluded from the analysis. The percentage of the genome with LOH was determined by the sum of the lengths of non-excluded LOH regions divided by the total length of the interrogable genome.

In equation form:

% genome with LOH=100*Σ(lengths of non-excluded LOH regions)/(total length of genome with SNP coverage−Σ(lengths of excluded LOH regions))

The total length of genome with SNP coverage for the T5 assay is 2.78E+09 base pairs.

Based on analysis of TCGA high-grade serous ovarian cancer dataset, a tumor tissue sample with at least 14% of genome with LOH is defined as high genomic LOH (LOH-positive). A tumor is HRD-positive if it is either BRCA-positive or LOH-positive, and HRD-negative only if it is both BRCA-negative and LOH-negative (Table 5). BRCA mutation analysis was determined based on screening and/or archival samples. Since genomic LOH may change over time, genomic LOH analysis was determined based on the screening samples.

TABLE 5
Definition of HRD-positive and HRD-negative populations

| | High genomic LOH: | Low genomic LOH: |
|---|---|---|
| | LOH-positive | LOH-negative |
| BRCA-positive | HRD-positive | HRD-positive |
| BRCA-negative | HRD-positive | HRD-negative |

### Results

Baseline and post-treatment target lesion scans from platinum-sensitive, relapsed, high-grade ovarian cancer patients to assess antitumor tumor activity of rucaparib in the different HRD subgroups were analyzed at various time points.

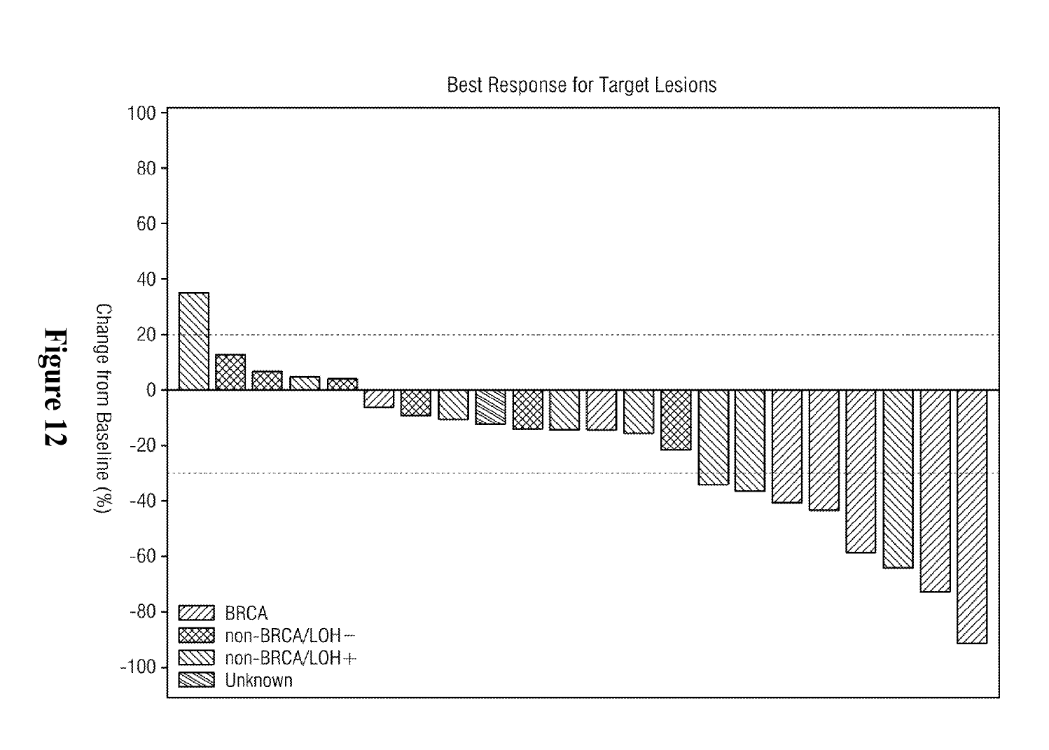

At time point A, 50 patients with platinum-sensitive, high-grade serous ovarian cancer had been sequenced and analyzed for BRCA mutations and genomic LOH. Of the 50 cases, 23 cases (46%) with BRCA1/2 mutations, 15 non-BRCA cases (30%) with high percentage of genome with LOH (non-BRCA/LOH+), and 12 non-BRCA cases (24%) with low percentage of genome with LOH (non-BRCA/LOH−). Tumor scans of baseline and post-treatment were available from 22 patients to assess antitumor tumor activity of rucaparib in the different HRD subgroups (FIG. 12). All 8 partial responders (PRs) identified were HRD-positive: 6 with BRCA mutations and 2 are non-BRCA/LOH+.

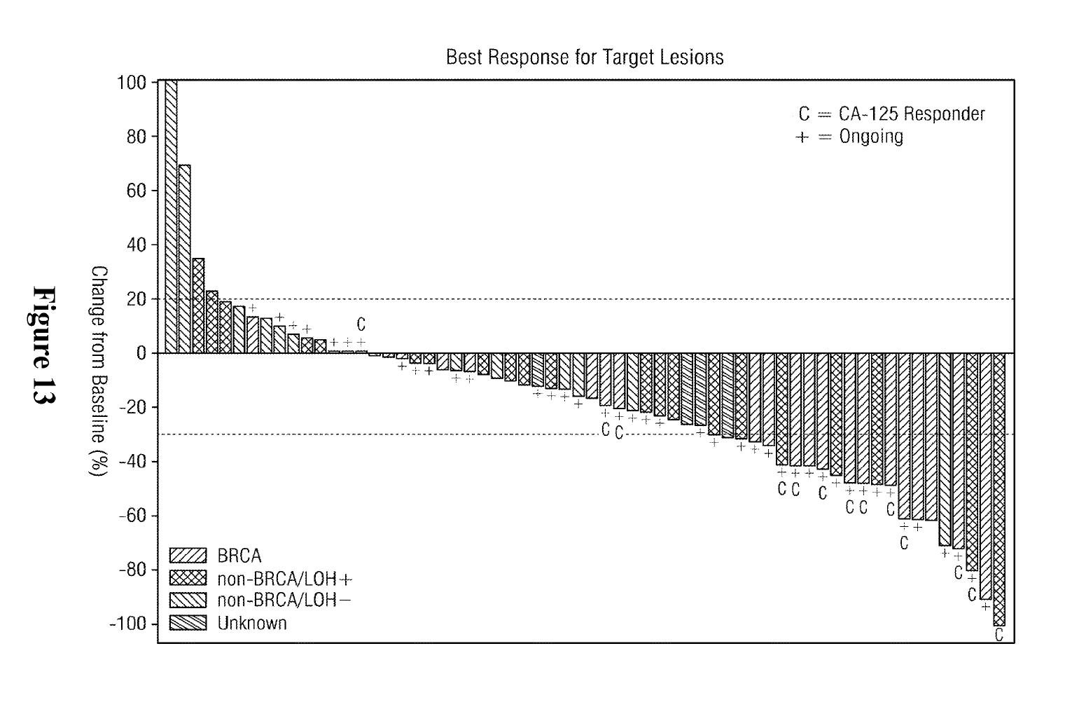

At time point B, 95 patients with platinum-sensitive, relapsed, high-grade serous ovarian cancer had been sequenced and analyzed for BRCA mutations and genomic LOH. Of the 95 cases, 26 cases (27%) with BRCA1/2 mutations, 39 non-BRCA cases (41%) with high percentage of genome with LOH (non-BRCA/LOH+), and 30 non-BRCA cases (32%) with low percentage of genome with LOH (non-BRCA/LOH−). Tumor scans of baseline and post-treatment were available from 61 patients to assess antitumor tumor activity of rucaparib in the different HRD subgroups (FIG. 13). Using the RECIST and GCIG CA-125 criteria for defining responders, the objective response rates (ORR) for the BRCA, non-BRCA/LOH+, and non-BRCA/LOH− subgroups were 68%, 28%, and 7%, respectively (Table 6).

TABLE 6
Definition of HRD-positive and HRD-negative populations

| | | RECIST ORR, | | RECIST & GCIG | |
|---|---|---|---|---|---|
| HRD Subgroup | # of Pts | % (n) | | CA-125 ORR, % (n) | |
| BRCA | 22 | 59 | (13/22) | 68 | (15/22) |
| Non-BRCA/LOH⁺ | 25 | 24 | (6/25) | 28 | (7/25) |
| Non-BRCA/LOH− | 14 | 7 | (1/14) | 7 | (1/14) |

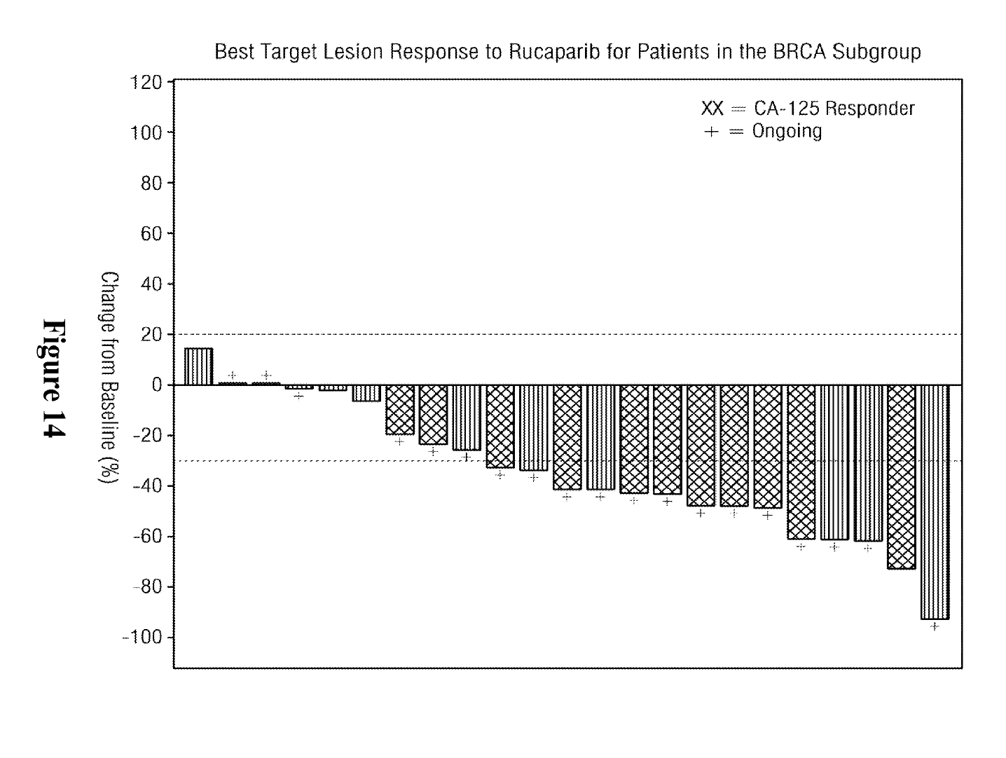

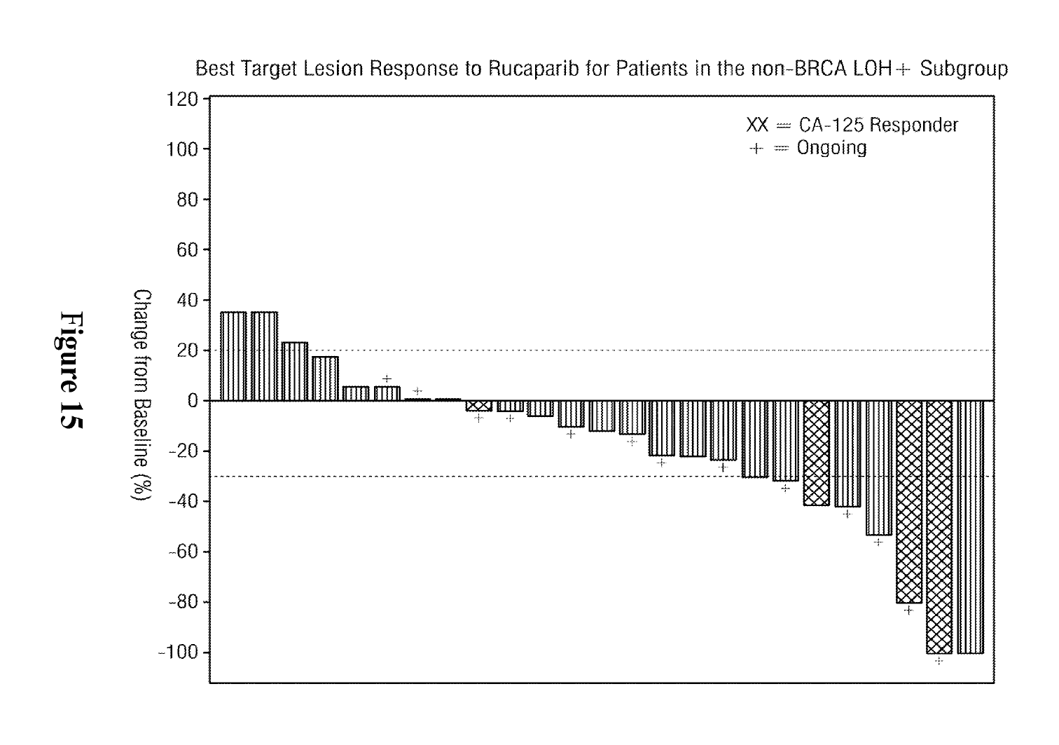

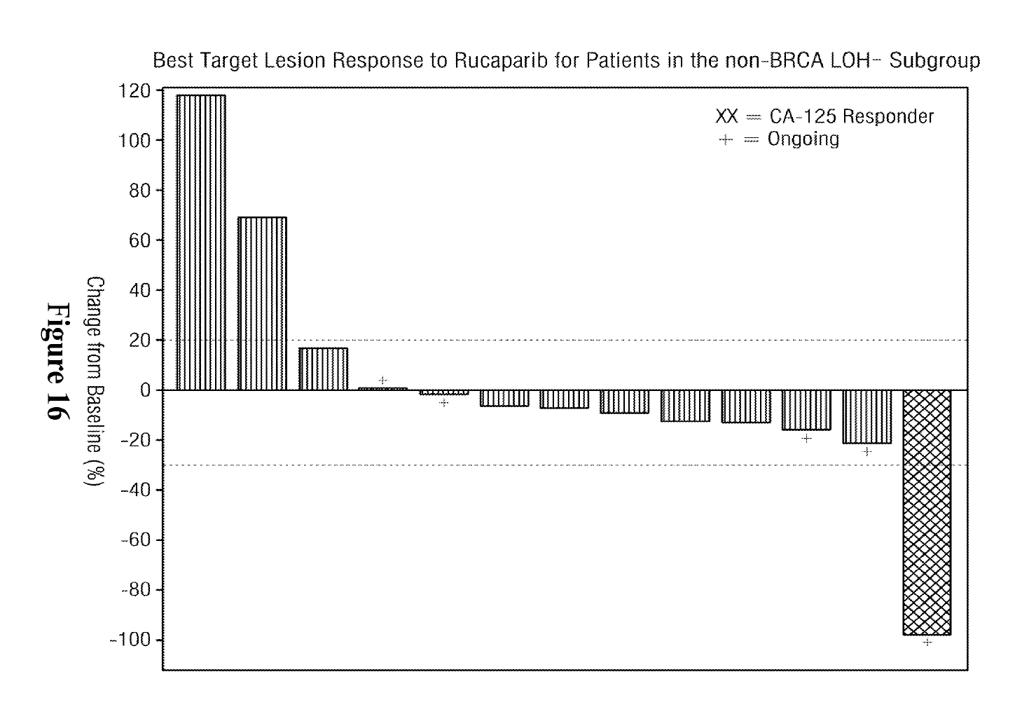

At time point C, target lesion scans of baseline and post-treatment were available from 61 with platinum-sensitive, relapsed, high-grade ovarian cancer patients to assess antitumor tumor activity of rucaparib in the different HRD subgroups: BRCA (FIG. 14), non-BRCA/LOH+(FIG. 15), non-BRCA/LOH− (FIG. 16). Using the RECIST and GCIG CA-125 criteria for defining responders, the overall response rates (ORR) for the BRCA, non-BRCA/LOH+, and non-BRCA/LOH− subgroups were 70%, 40%, and 8%, respectively (Table 7).

TABLE 7
RECIST and CA-125 overall response rates
(ORR) in different HRD subgroups

| | | RECIST ORR, | | RECIST & GCIG | |
|---|---|---|---|---|---|
| HRD Subgroup | # of Patients | % (n) | | CA-125 ORR, % (n) | |
| BRCA | 23 | 61 | (14/22) | 70 | (16/22) |
| Non-BRCA/LOH+ | 25 | 32 | (8/25) | 40 | (10/25) |
| Non-BRCA/LOH− | 13 | 8 | (1/13) | 8 | (1/13) |

The identification of non-BRCA/LOH+ patients who responded to rucaparib treatment exemplifies the clinical utility of the percentage of genome with LOH in predicting rucaparib sensitivity.

While preferred aspects of the present invention have been shown and described herein, it will be obvious to those skilled in the art that such aspects are provided by way of example only. Numerous variations, changes, and substitutions will now occur to those skilled in the art without departing from the invention. It should be understood that various alternatives to the aspects of the invention described herein may be employed in practicing the invention. It is intended that the following claims define the scope of the invention and that methods and structures within the scope of these claims and their equivalents be covered thereby.

1. A method for treatment of a cancer patient with a PARP inhibitor comprising:
a) receiving data from a computer system regarding the tumor of said cancer patient comprising:
i. the BRCA1 and BRCA2 mutation status, and
ii. the homozygous or heterozygous nature of a plurality of single nucleotides along each chromosome of the genome;
b) classifying said cancer patient, with the computer system, as being likely to respond to a PARP inhibitor if the data comprises:
iii. one or more deleterious mutations in BRCA1 or BRCA2, or
iv. a percentage of the genome having greater than about 10 percent LOH as determined by the sum of the lengths of each individual LOH region divided by the total genome length, wherein an LOH region is defined as the presence of homozygosity at multiple contiguous single nucleotides, but excludes whole chromosome LOH; and
c) administering a therapeutically effective amount of a PARP inhibitor to said cancer patient whose classification meets the criterion of step b).

2. The method of claim 1 wherein the PARP inhibitor is rucaparib.

3. The method of claim 1 wherein the cancer is breast cancer, ovarian cancer, or pancreatic cancer.

4. The method of claim 3 wherein the cancer is breast cancer.

5. The method of claim 4 wherein the breast cancer is triple negative breast cancer.

6. The method of claim 3 wherein the cancer is ovarian cancer.

7. The method of claim 6 wherein the ovarian cancer is high-grade serous ovarian cancer.

8. The method of claim 3 wherein the cancer is pancreatic cancer.

9. The method of claim 1 wherein the percentage of the genome having LOH as determined by the sum of the lengths of each individual LOH region divided by the total genome length is greater than about 11 percent, greater than about 12 percent, greater than about 13 percent, greater than about 14 percent, greater than about 15 percent, greater than about 16 percent, greater than about 17 percent, greater than about 18 percent, greater than about 19 percent, or greater than about 20 percent.

10. A method for treatment of a cancer patient with a PARP inhibitor comprising:
a) receiving data from a computer system regarding the tumor of said cancer patient comprising the homozygous or heterozygous nature of a plurality of single nucleotides along each chromosome of the genome;
b) classifying said cancer patient, with the computer system, as being likely to respond to a PARP inhibitor if the data comprises a percentage of the genome having greater than about 10 percent LOH as determined by the sum of the lengths of each individual LOH region divided by the total genome length, wherein an LOH region is defined as the presence of homozygosity at multiple contiguous single nucleotides, but excludes whole chromosome LOH; and
c) administering a therapeutically effective amount of a PARP inhibitor to said cancer patient whose classification meets the criterion of step b).

11. The method of claim 10 wherein the percentage of the genome having LOH as determined by the sum of the lengths of each individual LOH region divided by the total genome length is greater than about 11 percent, greater than about 12 percent, greater than about 13 percent, greater than about 14 percent, greater than about 15 percent, greater than about 16 percent, greater than about 17 percent, greater than about 18 percent, greater than about 19 percent, or greater than about 20 percent.
